(19) United States
(12) Patent Application Publication
(10) Pub. No.: US 2003/0099624 A1
(43) Pub. Date: May 29, 2003

(54) Administering bacilus laterosporus to increase poultry feed conversion and weight gain

(75) Inventor: Randolph S. Porubcan, Shakopee, MN (US)

Correspondence Address: Jeffrey L. Wendt, 600 Town Center One, 1450 Lake Robbins Drive, The Woodlands, TX, 77380, US

(73) Assignee: Microbes, Inc., The Woodlands, TX (US)

(21) Appl. No.: 10/128,186

(22) Filed: Apr. 23, 2002

Related U.S. Application Data
(60) Provisional application No. 60/303,196, filed on Jul. 5, 2001.

(51) Int. Cl. A61K 38/48; A23K 1/00

(52) U.S. Cl. 424/93.46; 426/53; 426/2

(57) ABSTRACT

Disclosed is a process for improving feed conversion and weight gain in poultry, including chickens, wherein Bacillus laterosporus, or any microorganism with a similarity index, based on its cellular fatty acid profile, of greater than 0.5 to Bacillus laterosporus (including Bacillus laterosporus strain CM-33 (ATCC Accession No. PTA-3952)) is administered to poultry. Strain CM-33 of Bacillus laterosporus was isolated from soil and has a similarity index of 76% to Bacillus laterosporus. The administration of strain CM-33 is preferably divided into daily doses of about 2.0 million colony forming units (cfu)/day and continued for about 40 days of the growth cycle. The cells or spores can be administered through the bird's drinking water or by other methods, including spraying them onto the bird's feed.

### CROSS REFERENCE TO RELATED APPLICATIONS

The present application claims priority priority under 35 U.S.C. 120 to Provisional Patent Application Serial No. 60/303,196 filed in the United States Patent and Trademark Office on Jul. 5, 2001 of which is incorporated herein by reference in its entirety.

### BACKGROUND OF THE INVENTION

Beneficial microorganisms can be fed to animals raised for human consumption, for the purpose of suppressing deleterious organisms or directly promoting the animal's growth. The Association of American Feed Control Officials (AAFCO) lists 41 probiotic microorganisms that are recommended for direct-feeding to animals. Official Publication (1989), Association of American Feed Control Officials Incorporated. 27 of these microorganisms (65% of the total) are classified as lactic acid-producing bacteria. Lactic acid-producing bacteria had been previously mentioned as having probiotic activity. However, only five species of Bacillus bacteria were on the list, and Bacillus laterosporus was not mentioned.

U.S. Pat. No. 5,045,314 discusses that certain strains of Bacillus laterosporus may control nematodes in ruminant animals, including goats. But such use was not mentioned for poultry, as they are not generally affected by nematode parasites. Twenty-seven strains of B. laterosporus are discussed, but only four, including strain ATCC 64, possessed nematicidal effect, and such effect was enhanced significantly by heat or enzyme treatment of the B. laterosporus, which caused activation of a specific toxin compound. There is, therefore, no suggestion or motivation to use any strain of Bacillus laterosporus for growth promotion in poultry, where nematodes are not considered a problem.

U.S. Pat. No. 5,283,059, Suzuki et al., discusses producing a stabilized Bacillus cereus animal feed preparation in pellet form that contains a complex carbohydrate such as cornstarch and Bacillus spores. The stabilizing effect is attained by heating the mixture of starch and Bacillus spores to 60-75° C. to gelatinize the starch, followed by a relatively complex preparation process involving spray drying, pulverization, granulation and pelleting.

U.S. Pat. No. 4,919,936, Iwanami et al., discloses a strain of Bacillus subtilis (FERM BP-1096) that improves weight gain in mice, piglets, calves, rainbow trout, layer chickens, and broiler chickens. The benefit to broiler chickens was reported to be 11 points of feed conversion (one point is considered a change of one unit in the second decimal position), but the feed contained up to 1×10⁶ cells/gram of Bacillus subtilis and was fed daily to the chickens for the entire growing period of 56 days. More effective feed conversion with less frequent administration of lesser amounts of bacteria is clearly desirable.

Other publications discussing probiotics include Fuller, R., Probiotics—The Scientific Basis, Chapman & Hall, 1992; and Doi, R. H. and McGloughin, M., Biology of Bacilli, Butterworth-Heinemann, 1992.

Recently, efforts have been made to market dried fecal contents from specific pathogen free chickens as probiotic products. The principle is to inoculate young chicks with such cultures (referred to as “competitive exclusion” or “CE cultures”) and create, within the chick's intestinal tract, a barrier of nonpathogenic fecal microorganisms that prevent or inhibit the colonization of pathogens, such as Salmonella. The problem with this approach is that particular probiotic CE cultures cannot be reproduced in a reliable manner in the feed. The fecal material may contain from 10-50 microbial strains, or more, and it is unknown which of these strains are protective or how to maintain them in the proper concentration from batch-to-batch.

U.S. Pat. Nos. 2,906,622 and 2,942,977 discuss growth stimulating agents. The '622 patent discusses use of two strains of bacillus subtilis, both of which produce subtilin, and other growth stimulating agents which exhibit the same antibiotic effects as subtilin and stimulate the growth of chicks and other animals when added to feed. The '977 patent discusses use of other strains of b. subtilis having the ability to: (i) produce growth stimulating factors, (ii) grow rapidly and efficiently under artificial culture conditions, and (iii) produce antibiotic agents, but not subtilin, bacitracin, or any of the other antibiotics which have been shown to be produced by antibiotic-producing members of the genus Bacillus. Neither patent discusses use of compositions comprising a non-lactic acid-producing bacteria having a similarity index of 0.5 or higher to Bacillus laterosporus.

To date, therefore, feeding of Bacillus laterosporus to chickens has not been disclosed or suggested.

### SUMMARY OF THE INVENTION

The invention includes administering to poultry the spores or live cells of Bacillus laterosporus, preferably strain CM-33 (deposited at the American Type Culture Collection (“ATCC”), P.O. Box 1549, Manassas Va. 20108, under accession No. PTA-3952), to cause one or more of: (i) weight gain, (ii) increased feed conversion to mass, and (iii) decreased mortality. Spores may be preferred as they may be more marketable than live cells, due to their longer shelf-life. Spores can be obtained by ultra-filtration, centrifugation, spray-drying, freeze-drying, or combinations thereof.

The spores of this CM-33 strain have a similarity index (based on cellular fatty acid analysis) of 0.763 to spores of Bacillus laterosporus, in an analysis wherein two samples with a similarity index above 0.5 are considered closely comparable. Administration of spores of strain CM-33 to broiler chickens at relatively low dose rates ranging from about 0.5 to about 8 million spores/bird/day, causes a substantial improvement in feed conversion and associated improvements in weight gain and mortality reduction. The hardiness and ease of spore production of strain CM-33 and similar strains permits administration by simple low cost methods, such as by incorporating it into feed, or adding it to drinking water.

The mechanism by which the CM-33 strain achieves its beneficial effects is not known. It is postulated that it may produce an antibiotic substance within the chicken's intestinal tract or in the litter environment, or it may colonize areas of the intestinal tract and competitively exclude deleterious microorganisms, or it may produce some nutrient factor that stimulates growth, or it may operate by some other mechanism.

The spores of strain CM-33 can be administered in suspension with drinking water, preferably chlorine-free water, which suspension may also include other additives and ingredients. It has been found to be further advantageous to sub-divide the total of colony forming units (“cfu”) into daily doses of about 2 million cfus per bird, over 40 days of the poultry growth cycle.

The invention and methods of making and using it are described further below.

### BRIEF DESCRIPTION OF THE DRAWING

FIG. 1 is a table that contains the cellular fatty acid (CFA) analysis for Bacillus laterosporus, strain CM-33.

### DETAILED DESCRIPTION OF THE INVENTION

A. Preparing a strain CM-33 Spore Suspension

In preparing spores of Bacillus laterosporus strain CM-33, a suitable microbiological medium is first selected. Both tryptic soy broth (TSB) and Schaeffer's Sporulation Medium, as referenced in Biology of Bacilli (Doi, et al. Butterworth-Heinemann, 1992), are suitable growth media. The medium is sterilized in baffled Erlenmeyer flasks at 121° C. under 15 psig for 30 minutes, or until rendered sterile. The Erlenmeyer flasks should be under-filled to optimize aeration during shaking. About 200 ml of medium is preferred for use in a 4 liter Erlenmeyer flask.

The flask is fitted with a sterile filter cap that allows the contents to breath without becoming contaminated. The sterile medium is inoculated from a slant culture on tryptic soy agar, preferably by having a slant medium with good colony growth melted and poured into the Erlenmeyer flask. The inoculated medium is then shaken on a rotary orbital shaker at 100-200 rpm at 32° C. for 48 hours. The CM-33 strain will typically be 90% sporulated within 48 hours, displaying a viable spore count of about 10⁸/ml. The resulting spore suspensioncan be administered to poultry without further preparation. If the spore suspension is not used within one week of preparation, it should be refrigerated at 5° C. until ready for use. Spore suspensions held at 5° C. have a half-life of about two months.

The Bacillus laterosporus spores may also be purified or concentrated using methods such as ultra-filtration, centrifugation, spray-drying or freeze-drying to generate a packaged product. Such preparations may be more marketable due to their longer shelf-life, but freshly-prepared suspensions should be equally efficacious.

The spores may be present in a composition that includes water, or water and additives and excipients that do not have a deleterious effect on the action of the spores, or water, additives and excipients and other ingredients conventionally used in spore preparations. The composition may, optionally, also include other feed additives, including nutrient organic compounds and trace minerals or vitamins, antibiotics, growth factors and adjuvants. The composition may also include, optionally, a mixture of vegetative cells of non-lactic acid-producing bacteria, preferably having a similarity index of 0.5 or higher compared to Bacillus laterosporus type strain.

Preparation of spores of other strains of Bacillus laterosporus which improve growth or feed conversion in poultry may be prepared by similar methods. Preparation of vegetative cells of strain CM-33 or other strains may be prepared by methods well known in the art.

B. Characterization of Strain CM-33

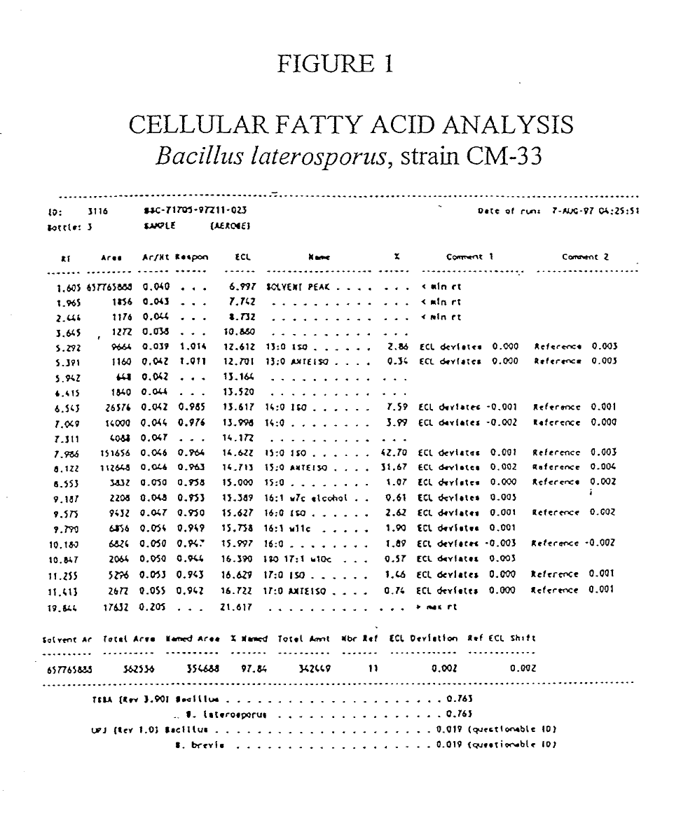

Bacillus laterosporus, strain CM-33 has been characterized morphologically and physiologically and these results are summarized in Table A below and in FIG. 1.

TABLE A
Bacillus laterosporus strain CM-33
Morphological Data:

Gram positive rod-slender and motile, length 2-6 um, width < 1 um.
Sporangium-not swollen.
Endospores are oval and cradled by canoe-shaped parasporal body.
Endospores located sub-middle.
Rods may curve and become spindle-shaped when they produce
endospores.

Physiological Data: (+= positive, n = negative)

| Parameter | Result |
|---|---|
| Anaerobic growth | + |
| Catalase | + |
| Growth at 65° C. | n |
| Starch hydrolysis | n |
| Gelatin liquification | n |
| Casein hydrolysis | + |
| Glucose (acid, no gas) | + |
| Mannitol | n |
| Glycerol | n |
| Arabinose | n |
| Xylose | n |
| Citrate utilization | n |
| Growth at < pH 5.7 | n |
| Growth in 7% NaCl | n |
| Nitrate reduction | + |
| Methyl red test | + |
| Oxidase | + |
| Trehalose (acid, no gas) | + |
| Lactose | n |
| Sucrose | n |
| Fructose | + |
| Urea hydrolysis | n |
| Esculin hydrolysis | + |
| Arginine utilization | + |
| Phenylalanine deamination | n |

FIG. 1 displays the cellular fatty acid (CFA) analysis for Bacillus laterosporus, strain CM-33, listing the retention times (RT column) and areas under the peaks (area column) for the fatty acids present in an extract of the Bacillus cells. The CM-33 strain was sub-cultured twice and analyzed using the MIDI/Hewlett Packard Microbial Identification System (MIS). The data was obtained on high-resolution gas chromatography and the analysis, taken in total, represents a biochemical fingerprint of the organism. The profile obtained was compared to the profile of the type strain for that species by computer analysis. The similarity index shown at the bottom of the profile (0.763) represents the percent agreement with Bacillus laterosporus type strain. A Similarity Index of 0.500 or higher is considered a close comparison.

C. Administration to Poultry

The spores or cells of the Bacillus laterosporus strain CM-33 can be administered to poultry by a number of methods, including by spraying an aqueous suspension thereof onto poultry feed, or mixing it with the poultry drinking water. In the latter method, measured quantities of the aqueous spore suspension are added, daily, to the watering system, either manually or by metering pump. The dose of spores per bird can be varied as the bird grows, although it is preferred to feed a standardized dose (fixed number of spores/bird/day) for a specific number of days during the growth period. It is not necessary to feed the spores to the poultry from day one (i.e., chick placement) through harvest (day 42-52). For purposes of economy, feeding of the spores can begin from about day 10 to about day 23 and proceed to harvest. In a preferred embodiment, spores are fed starting at about day 10 and continued daily for 40 days. If harvest is projected to be less than 50 days, the spore feeding is started at a corresponding number of days earlier so that the birds are fed spores for 40 days. The preferred dose range for Bacillus laterosporus, strain CM-33, spores is from about 0.5 million cfu/bird/day to about 8 million cfu/bird/day (representing a total dose administration of 20 to 320 million cfu over the life of a bird), and more preferably about 2 million cfu/bird/day. The cfu determination was made by plate count on tryptic soy agar incubated for 72 hours at 32° C.

The spores are preferably administered mixed with commercial, nutrient enriched, corn-soy diets. The simultaneous use of growth-promoting antibiotics may be contra-indicated, but may be acceptable, depending on the specific antibiotic and the quantities used. For example, coccidiostats (such as that known under the trade designation “Coban”) do not adversely affect the CM-33 spores and can be used simultaneously. If it is necessary to use antibiotics which inhibit CM-33 strain in the starter diet, the spores can be started when the switch to grower feed (without antibiotics) is made.

For chickens, it is preferred that 2 million cfu of CM-33 spores are fed to each bird each day for at least 40 days, typically from day 10 to day 50. When 2 million spores are fed daily for 40 days, a total dose of 80 million spores per bird is administered. This relatively low dose results in a substantial benefit in growth (19 points of feed conversion were achieved with this dosage, see Example I below, where each point of feed conversion represents a 0.01 increase in F/G (explained below)). Lactic acid-producing bacteria have been administered to chickens at total dose rates in excess of 500 million to 1 billion cfu, with less improvement in feed conversion (see Fuller, R., Probiotics—The Scientific Basis, Chapman & Hall, 1992).

Compositions of strain CM-33 suitable for poultry feed may include a variety of optional ingredients, e.g., vitamins and minerals, growth factors, adjuvants, and the like. Although these are not required, and may not increase weight gain or decrease food consumption. Vitamin additives may also be included, for example, one or more of pantothenic acid, pyridoxine, riboflavin, thiamin, 25-hydroxy vitamin A, B12, C,D, E, biotin, choline, folacin, niacin, and vitamin K. Mineral additives may be included, for example, one or more of magnesium, potassium, sodium, copper, iodine, iron, manganese calcium, phosphorous, selenium, chlorine and chromium pincolinate. The concentration of the vitamins and minerals will depend upon the animal being treated but, in general, will be between about 0.01% and about 5% by weight of the feed dry matter.

Compositions including strain CM-33 may also include other substances, for example, one or more of keratinocyte growth factor, nerve growth factor, vascular endothelial growth factor, somatotropin, insulin-like growth factor (IGF-I or IGF-II), epidermal growth factor, transforming growth factor, bombesin, fibroblast growth factor, granulocyte-macrophage colony stimulating factor and erythropoietin. Such compositions may also contain a steroid or hormone including one or more of estrogens, glucocorticoids, insulin, glucagon, gastrin, calcitonin and somatotropin. The high moisture material may further contain an antibiotic including one or more of lincomycin, virginiamycin, bacitracin, BMD (bacitracin methylenedisalicylate), and others. Such compositions may also include one or more of a natural or synthetic antioxidant such as BHA (butylated hydroxyanisole), vitamin C or glutathioneethoxyquin, tocopherol, BHT (butylated hydroxytoluene); a receptor, transfer factor, chelator or complexing agent which modifies release rates of nutrients or other bioactive compounds; an immunoactive agent such as immunoglobulins, antigens, killed cells, attenuated strains, toxins, adjuvants, cytokines, vaccines and other immunomodulators; or a palatability modifier or intake regulator such as food coloring, grit, oyster shell, whole seeds or grains. These substances can be used alone or in combination with one another. The concentration of these additives will depend upon the animal being treated but, in general, if used at all, will be included at about 0.0001% and about 10% by weight of the dry matter, and more preferably between about 0.001% and about 7.5%, most preferably between about 0.01% and about 5%.

Substances useful as palatability modifiers or intake regulators in addition to those mentioned above include triglycerides, clonidine, gums and hydrolyzed gums such as guar gum, xanthan gum or algin; fish products such as fishmeal and fish oils; spices such as sage, thyme, cloves, and the like; gastrin antagonists; cholecystokinin antagonists; amino acids such as methionine, tyrosine, phenylalanine, and the like; naloxone; pancreatic polypeptide; norepinephrine; melatonin antagonists; and thyroid hormones such as thyroxine, T3, T4, and the like.

Adjuvants that can be incorporated into such compositions can be of several different types, e.g., oils, peptides, polypeptides, microbiologically-derived substances, lectins, polysaccharides, and proteins, and various nucleic acids. Microbiologically-derived substances include materials produced by, or which are cellular components of, microorganisms such as bacteria, fungi including yeasts, and the like.

Vaccines useful for administration to poultry with the strain CM-33 include those effective against common diseases in poultry such as Newcastle's Disease, Marek's Disease, infectious bursal disease, infectious bronchitis, enteritis and coccidiosis. These vaccines include, for example, Newcastle's vaccine, Marek's Disease vaccine, infectious bursal disease vaccine, infectious bronchitis vaccine and CocciVac.RTM. Such vaccines can be administered to young birds orally, via yolk sac injection, subcutaneously, in ovo, or via inhalation by mist or spray.

Strains of Bacillus laterosporus other than CM-33 could be prepared and administered by similar methods and in similar compositions, with similar additives, to those described above.

The benefit of administration of Bacillus laterosporus to poultry is exemplified below.

### EXAMPLE I

### Effects of Strain CM-33 Administration to Chickens

Bacillus laterosporus strain CM-33 spores were prepared in TSB shake flasks (200 ml in a 4 liter baffled flask) inoculated from a melted TSA slant culture and incubated for 48 hours at 32° C. with constant 100 rpm orbital agitation. This resulted in a spore suspension containing 200 million viable spores per ml. The spore suspension was administered to broiler chickens at the rate of 2 million cfu/bird/day beginning at day 10 of a bird's life and continuing through day 50 at which time the birds were harvested. This represents a total dose of 2 million cfu/day×40 days, or 80 million total cfus during the life of the bird. 2000 birds, 1000 controls and 1000 treated, were involved in the test. An antibiotic-free corn-soy ration was fed with the additive Coban incorporated as the coccidiostat. Spore suspensions were manually applied by pipette to the bird's water supply, with adjustment every fifth day for any increased water consumption. Mortality counts were recorded daily and feed conversion and final weights were computed at harvest. The results are presented in Table I.

TABLE I
Results-Example I

| | Test | Control |
|---|---|---|
| # chickens | 1,000 | 1,000 |
| Avg. mortality | 6.8% | 9.5% |
| Avg. final weight | 2.20 kg | 2.06 kg |
| F/G* | 2.12 | 2.31 |

Feed conversion improvement with test = 19 points.
*F/G denotes the feed provided a bird, in kg, over the bird's growth, in kg.

### EXAMPLE II

### Low Dose Study

Bacillus laterosporus, strain CM-33, spores were prepared in TSB shake flasks (200 ml in a 4 liter baffled flask) inoculated from a melted TSA slant culture and incubated for 48 hours at 32° C. with constant 100 rpm orbital agitation. This resulted in a spore suspension containing 150 million viable spores per ml. The spore suspension was administered to broiler chickens at the rate of 0.5 million cfu/bird/day beginning at day 10 of a bird's life and continuing through day 50, at which time the birds were harvested. This represents a total dose of 0.5 million cfu/day for 40 days, or a total of 20 million cfus during the life of a bird. 2000 birds were involved—1000 controls and 1000 treated. An antibiotic-free corn-soy ration was fed with Coban. Spore suspensions were manually applied by pipette to the bird's water supply, with adjustment every fifth day for any increased water consumption. Mortality counts were recorded daily, and feed conversion and final weights were computed at harvest. The results are presented in Table II.

TABLE II
Results-Example II

| | Test | Control |
|---|---|---|
| No. chickens | 1,000 | 1,000 |
| Avg. mortality | 8.6% | 9.8% |
| Avg. final weight | 2.10 kg | 2.02 kg |
| F/G | 2.28 | 2.34 |

Feed conversion improvement with test = 6 points.

### EXAMPLE III

### High Dose Study

Bacillus laterosporus strain CM-33 spores were prepared in TSB shake flasks (200 ml in 4 liter baffled flasks) inoculated from a melted TSA slant culture and incubated for 48 hours at 32° C. with constant 100 rpm orbital agitation. This resulted in a spore suspension containing 250 million viable spores per ml. The spore suspension was then administered to broiler chickens at the rate of 8 million cfu/bird/day beginning at day 10 of a bird's life, and continuing through day 50, at which time the birds were harvested. This represents a total dose of 8 million cfu/day for 40 days, or a total of 320 million cfus during the life of a bird. 2000 birds were involved—1000 controls and 1000 treated. An antibiotic-free corn-soy ration was fed with Coban. Spore suspension was manually applied by pipette to the bird's water supply, with adjustments every fifth day for any increased water consumption. Mortality counts were recorded daily, and feed conversion and final weights were computed at harvest. The results are presented in Table III.

TABLE III
Results-Example III

| | Test | Control |
|---|---|---|
| No. chickens | 1,000 | 1,000 |
| Avg. mortality | 6.2% | 9.0% |
| Avg. final weight | 2.21 kg | 2.08 kg |
| F/G | 2.08 | 2.28 |

Feed conversion improvement with test = 20 points.

### EXAMPLE IV

### Shortened Dosing Period

Bacillus laterosporus strain CM-33 spores were prepared in TSB shake flasks (200 ml in 4 liter baffled flasks) inoculated from a melted TSA slant culture and incubated for 48 hours at 32° C. with constant 100 rpm orbital agitation. This resulted in a spore suspension containing 190 million viable spores per ml. The spore suspension was administered to broiler chickens at the rate of 2 million cfu/bird/day beginning at day 23 of a bird's life and continuing through day 48, at which time the birds were harvested. This represents a total dose of 2 million cfu/day for 25 days, or 50 million cfus administered during the life of the bird. 1,300 birds were involved—700 controls and 600 treated. An un-medicated corn-soy ration was fed to the birds. The spore suspension was manually applied by pipette to the bird's water supply, with adjustments every fifth day for any increased water consumption. Mortality counts were recorded daily, and feed conversion and final weights were computed at harvest. The results are presented in Table IV.

TABLE IV
Results-Example IV

| | Test | Control |
|---|---|---|
| No. chickens | 600 | 700 |
| Avg. mortality | 6.7% | 9.3% |
| Avg. final weight | 2.21 kg | 2.06 kg |
| F/G | 2.30 | 2.46 |

Feed conversion improvement with test = 16 points.

### EXAMPLE V

### Dry Feed Application

Bacillus laterosporus strain CM-33 spores were prepared in TSB shake flasks (200 ml in 4 liter baffled flasks) inoculated from a melted TSA slant culture and incubated for 48 hours at 32° C. with constant 100 rpm orbital agitation. This resulted in a spore suspension containing 240 million viable spores per ml. The spore suspension was sprayed onto broiler chicken feed one hour prior to feeding the chickens, providing an amount equal to 2 million spores/bird/day administered with the first feeding in the morning. The spore suspension was diluted in sufficient chlorine-free water to facilitate even distribution over the feed and assure that all treated chickens received the specified dose. The amount of water used was about 1.5%, by weight, of the feed. The feed, so treated with spores, was fed from day 10 of the bird's life to day 50, at which time the birds were harvested. This represents a total dose of 2 million cfu/day for 40 days, or 80 million cfus during the life of the bird. There were 2000 birds involved with the test—1000 controls and 1000 treated. An antibiotic-free corn-soy ration was fed with Coban. Mortality counts were recorded daily, and feed conversion and final weights were computed at harvest. The results are presented in Table V.

TABLE V
Results-Example V

| | Test | Control |
|---|---|---|
| No. chickens | 1,000 | 1,000 |
| Avg. mortality | 6.2% | 9.0% |
| Avg. final weight | 2.21 kg | 2.08 kg |
| F/G | 2.08 | 2.28 |

Feed conversion improvement with test = 20 points.

### EXAMPLE VI

### Trial Using Commercial Production

Bacillus laterosporus, strain CM-33, spores were prepared in a 2,000 liter fermentor using Schaeffer's Sporulation Medium. The medium was sterilized at 121° C. for 30 minutes, cooled to 32° C. and inoculated at 1% by volume with a shake flask culture of Bacillus laterosporus, strain CM-33. The fermentor was then agitated at 150 rpm while sterile air was sparged into the liquid culture at a rate of 500 liters of air per minute. Temperature was maintained at 32° C. for 45 hours. This resulted in a spore suspension containing 300 million viable spores per ml. The spore suspension was then administered to broiler chickens at the rate of 2 million cfu/bird/day, beginning on day 12 of a bird's life and continuing through day 52, at which time the birds were harvested. This represents a total dose of 2 million cfu/day for 40 days, or 80 million cfus during the life of the bird. 8,100 birds were involved with the test—3,200 controls and 4,900 treated. A corn-soy ration containing Coban was fed to the birds. The spore suspension were applied by metering pump to the bird's water supply, with adjustments daily for increased water consumption. Mortality counts were recorded daily, and feed conversion and final weights were computed at harvest. The results are presented in Table VI.

TABLE VI
Results-Example VI

| | Test | Control |
|---|---|---|
| No. chickens | 4,900 | 3,200 |
| Avg. mortality | 2.67% | 3.69% |
| Avg. final weight | 2.60 kg | 2.50 kg |
| F/G | 1.90 | 2.06 |

Feed conversion improvement with test = 16 points.

A summary of the results of Examples I-VI are presented in in Table VII.

TABLE VII
Summary Table for Examples I-VI
(C = Control, T = Test)

| EXAMPLE | Mortality- | Weight- | F/G- | Mortality- | Weight- | F/G- |
|---|---|---|---|---|---|---|
| # | C | C | C | T | T | T |
| I | 9.5 | 2.06 | 2.31 | 6.8 | 2.20 | 2.12 |

Preferred Dosing—80 million cfu total dose—F/G improvement =
19 points

| | | | | | | |
|---|---|---|---|---|---|---|
| II | 9.8 | 2.02 | 2.34 | 8.6 | 2.10 | 2.28 |

Low Dose Study—20 million cfu total dose—F/G improvement = 6 points

| | | | | | | |
|---|---|---|---|---|---|---|
| III | 9.0 | 2.08 | 2.28 | 6.2 | 2.21 | 2.0 |

High Dose Study—320 million cfu total dose—F/G improvement =
20 points

| | | | | | | |
|---|---|---|---|---|---|---|
| IV | 9.3 | 2.06 | 2.46 | 6.7 | 2.21 | 2.30 |

Shortened Dosing—50 million cfu total dose—F/G improvement =
16 points

| | | | | | | |
|---|---|---|---|---|---|---|
| V | 8.8 | 2.14 | 2.27 | 6.1 | 2.24 | 2.16 |

Dry Feed Application—80 million cfu total dose—F/G improvement =
11 points

| | | | | | | |
|---|---|---|---|---|---|---|
| VI | 3.69 | 2.50 | 2.06 | 2.67 | 2.60 | 1.90 |

Commercial Trial—80 million cfu total dose—F/G improvement =
16 points

It can be seen that in Example I, the relative benefit (on the basis of F/G improvement for cfus administered) of the administration of strain CM-33 is maximized by the application, in chlorine-free drinking water, of 80,000,000 cfu/bird, beginning at day 10 and dosed daily at 2,000,000 cfu/bird for 40 days.

The invention includes numerous variations, modifications and alterations of the embodiments and methods described in the specification above, and the scope of the invention is not defined or limited by this specification or by the examples, but is defined only in the claims that follow, and includes all equivalents of the subject matter of the claims.

What is claimed is:

1. A process for improving feed conversion and weight gain in poultry comprising administering Bacillus laterosporus or any microorganism with a similarity index, based on its cellular fatty acid profile, of greater than 0.5 to Bacillus laterosporus, to poultry in an amount effective to accomplish said improvements.

2. The process of claim 1 wherein spores of Bacillus laterosporus strain CM-33 (ATCC Accession No. PTA-3952 ) are administered to poultry.

3. The process of claim 2 wherein at least about 20,000,000 colony forming units of the spores are administered over the life of a bird.

4. The process of claim 3 wherein about 80,000,000 colony forming units of the spores are administered over the life of a bird.

5. The process of claim 4 wherein the spores are administered for forty days at 2,000,000 colony forming units per bird per day.

6. The process of claim 5 wherein the bird is harvested at the completion of said administration.

7. The process of any of claims 1 to 6 wherein the spores are administered by incorporating them into the bird's feed ration prior to feeding.

8. The process of any of claims 1 to 6 wherein the spores are administered by adding it to the bird's drinking water or by spraying it, as an aqueous suspension, onto the bird's feed ration prior to feeding.

9. The process of claim 8 wherein the spores are administered to a 10 day old bird by adding it to the bird's drinking water, and said administration is continued for forty days.

10. The process of claim 9 further including feeding the bird a corn-soy ration which includes a coccidiostat.

11. A composition comprising substantially purified spores or live cells of Bacillus laterosporus strain CM-33 (ATCC Accession No. PTA-3952).

12. The composition of claim 11 in a water suspension, wherein the water is substantially chlorine-free.
